(19) United States
(12) Patent Application Publication
(10) Pub. No.: US 2023/0320598 A1
(43) Pub. Date: Oct. 12, 2023

(54) INTRA-BEAT BIOMARKER FOR ACCURATE BLOOD PRESSURE ESTIMATIONS

(71) Applicant: THE REGENTS OF THE UNIVERSITY OF CALIFORNIA, Oakland, CA (US)

(72) Inventors: Michelle Khine, Irvine, CA (US); Arash Abiri, Irvine, CA (US)

(21) Appl. No.: 18/296,637

(22) Filed: Apr. 6, 2023

Related U.S. Application Data
(60) Provisional application No. 63/328,022, filed on Apr. 6, 2022.

(51) Int. Cl. A61B 5/021; A61B 5/00

(52) U.S. Cl. CPC A61B 5/02125; A61B 5/7278; A61B 2560/0223

(57) ABSTRACT

A method comprising receiving a blood pressure waveform from a sensor and deriving initial values. The method may further comprise deriving a diastolic transit time (DTT) value and a plurality of additional properties from the waveform. The method may further comprise calculating a calibration factor based on the DTT values and the additional properties. The method may further comprise calculating estimated DBP values based on the calibration factor, the DTT values, and the additional properties. The method may further comprise deriving an offset value based on a difference between the estimated DBP values and the raw DBP values, adjusting the blood pressure waveform based on the offset to generate an adjusted blood pressure waveform, and outputting the adjusted blood pressure waveform.

### CROSS-REFERENCES TO RELATED APPLICATIONS

This application is a non-provisional and claims benefit of U.S. Provisional Application No. 63/328,022 filed Apr. 6, 2022, the specification of which is incorporated herein in its entirety by reference.

### FIELD OF THE INVENTION

The present invention is directed to continuous, non-invasive, beat-to-beat blood pressure monitoring through measurement of diastolic transit time with a single sensor.

### BACKGROUND OF THE INVENTION

Blood pressure (BP) is an important physiologic metric that provides insights into a patient's cardiac function, volume status, organ perfusion, and overall hemodynamic stability. Most commonly, BP is measured intermittently at the arm using a non-invasive sphygmomanometer (i.e., arm cuff). In high-acuity medical settings, such as the operating room (OR) and intensive care unit (ICU), continuous BP monitoring is achieved via an invasive arterial catheter (A-line) placed in a peripheral artery. While the A-line allows for the detection of sudden hemodynamic changes since A-lines are highly invasive and associated with a number of medical complications, including hematoma, arterial thrombosis, and infection, their use is often limited to patients who are “high risk.” In the U.S., it is estimated that only 36% of critically ill patients in the ICU receive an A-line. Given the arm cuff's relatively low precision compared to the A-line, and its tendency to overestimate low BPs and underestimate high BPs, its use as the sole instrument for measuring BP in most patients results in undetected critical hemodynamic changes that may have otherwise influenced patient care. Additionally, recent studies have correlated continuous BP patterns with cardiovascular outcomes; for example, the variability in beat-to-beat BP measurements can be used to assess important physiological parameters, such as vascular tone, fluid responsiveness, and sympathetic autoregulation.

For this reason, continuous noninvasive BP (CNIBP) monitoring has garnered increasing interest over the past several decades yet remains an elusive unmet need. One of the first discovered CNIBP techniques was the volume-clamp method, which has been implemented in several commercial devices, including ClearSight® (Edwards Lifesciences, Irvine, CA, USA), Caretaker® (Caretaker Medical NA, Charlottesville, VA, USA), and CNAP® (CNSystems, Graz, Austria). Despite meeting the accuracy guidelines (mean average error≤5 mmHg and standard deviation<8 mmHg) cited by the Association of Advancement of Medical Instrumentation (AAMI) and U.S. Food and Drug Administration (FDA), their widespread use has been largely limited due to their bulkiness, high cost, and inconvenient form factor.

Non-invasive blood pressure monitoring technologies, regardless of sensing modality (PPG, tonometry) all face significant drift and noise issues that prevent long-term accurate continuous blood pressure monitoring. Some commercial devices have to recalibrate every minute to try to overcome these issues, which is impractical and has stymied adoption.

Photoplethysmography (PPG)-based devices have attracted attention for their smaller form factor compared to volume-clamp devices. As they are susceptible to interference from ambient light, changes in skin (applanation) pressure, and low-frequency baseline wander, PPG-based devices rely on a number of pre-processing steps, and often include moving average filters, frequency filters, and other noise-reduction techniques, such as discrete wavelet transformation (DWT) or empirical mode decomposition (EMD). Furthermore, since PPG requires high applanation pressures to achieve adequate morphologic resolution, many devices have transitioned to using the PPG signal's temporal dimension via pulse wave velocity (PWV) to more reliably estimate BP. PWV can be calculated from either the pulse transit time (PTT) or pulse arrival time (PAT). The former is defined as the time taken by a pressure wave to travel between two arterial sites and can be calculated by using two synchronized PPG signals at two different peripheral sites. The latter is defined as the PTT interval plus the pre-ejection period, which represents the delay between electrical depolarization of the left ventricle and the onset of ventricular ejection. By using an additional nearby electrocardiogram (ECG) device, PAT was developed to overcome the significant challenge of calibrating two anatomically distant PPG devices.

Many studies have described methods for estimating BP using PTT or PAT. Two of the most cited algorithms in this area, developed under the basis of the Moens—Kortweg equation, proposed quick calibration of PTT/PAT values by using a single reference BP value (e.g., arm cuff). While both studies had initially shown satisfactory correlations to the radial A-line, it was found that the proposed systems required recalibration every 4 minutes and tracked BP changes poorly. Furthermore, in the context of vasoactive drugs, it was suggested that the calibration interval be further reduced to 60 beats. Conversely, the algorithm demonstrated improved BP tracking capabilities but required recalibration every 45 seconds. Indeed, since their inception, numerous variations of these algorithms have been developed to improve accuracy and reduce calibration dependency. Moreover, pulse wave decomposition analysis and more complex models (e.g., via machine learning) have been implemented to improve measurement performance. However, the external validity of these algorithms and their ability to perform in different clinical contexts has been difficult to assess, since most studies do not report their calibration intervals and data's hemodynamic ranges.

Prior systems have proposed a single intra-beat PPG signal feature coined slope transit time (STT) as a proxy alternative of PTT for tracking blood pressure. While this approach was not validated against the A-line or an FDA-cleared device, a recent study demonstrated its ability to estimate systolic blood pressure (SBP) in the context of artificially generated baseline wander. Most recently, a normalized STT (NSTT) was proposed, which aimed to improve the stability of STT with normalization by PPG height. Among 40 hemodynamically stable subjects, this approach demonstrated comparable results to an FDA-cleared tonometry device. However, since their technique relied on performing linear regression on their recorded waveform features, future investigations using clearly defined, distinct training and validation datasets are still warranted in order to assess its accuracy.

The advent of Microelectromechanical System (MEMS) technology has introduced the prospect of using small, wearable capacitive pressure (CAP) sensors for CNIBP monitoring. Over the years, CAP sensors have grown increasingly popular due to their convenient form factor, high spatial resolution, quick response times, and low power consumption requirements. Unlike PPG- and oscillometry-based devices, CAP sensors detect pulsatile flow from the artery by measuring the changes in capacitance that result from compression and expansion of the soft dielectric layer. Thus, similar to arterial tonometry, CAP sensors are placed directly over the artery, and with the use of an initial arm cuff measurement, can be calibrated to measure beat-to-beat BP. Although CAP sensors were previously limited by their low sensitivities, recent advancements in sensor design have largely overcome this challenge.

While studies have previously demonstrated the potential for CAP sensors to correlate well with measurements from the A-line, similar to PPG, the quality of the data is highly dependent on applanation pressures. Additionally, due to respiratory variations and the stochastic behavior of the viscoelastic polymer sensors, they are highly susceptible to baseline wander. Thus, as with PPG signals, scientists have had to employ a variety of filters to eliminate this low-frequency baseline wander. While seemingly successful, these filters have been primarily tested on short signal segments where there were little to no changes in BP. For example, studies have often employed high-pass frequency filters with cut-offs of 0.25-0.5 Hz to reduce baseline wander; however, this approach may impede the ability to perceive slow physiological drifts in BP that fall below this frequency range. Moreover, studies have not examined how these filters may alter interpretations of blood pressure variability (BPV). Therefore, the validity and safety of using such filters on physiological signals that are intended to inform medical decisions remain largely unknown. Thus, there exists a present need for a unique and generalizable algorithm to accurately estimate BP parameters using a novel intra-beat biomarker, with significant implications in enhancing CNIBP technologies and overcoming challenges that have heretofore hindered their wide-scale adoption and usefulness.

### BRIEF SUMMARY OF THE INVENTION

It is an objective of the present invention to provide systems and methods that allow for continuous, non-invasive, beat-to-beat blood pressure monitoring, as specified in the independent claims. Embodiments of the invention are given in the dependent claims. Embodiments of the present invention can be freely combined with each other if they are not mutually exclusive.

The present invention features a system for continuous, non-invasive, beat-to-beat blood pressure monitoring of a subject. In some embodiments, the system may comprise a sensor coupled to the subject for measuring a blood pressure waveform. The system may further comprise a computing device communicatively coupled to the sensor capable of receiving the blood pressure waveform from the sensor and deriving initial values from the blood pressure waveform. The computing device may further be capable of deriving a diastolic transit time (DTT) value and a plurality of additional properties from the blood pressure waveform. The computing device may further be capable of calculating a calibration factor based on the DTT values and the plurality of additional properties. The computing device may further be capable of calculating estimated DBP values based on the calibration factor, the DTT values, and the plurality of additional properties. The computing device may further be capable of deriving an offset value based on a difference between the one or more estimated DBP values and the one or more raw DBP values, adjusting the blood pressure waveform based on the offset to generate an adjusted blood pressure waveform, and outputting the adjusted blood pressure waveform.

The present invention features a method for continuous, non-invasive, beat-to-beat blood pressure monitoring of a subject. In some embodiments, the method may comprise receiving a blood pressure waveform from a sensor and deriving initial values from the blood pressure waveform. The method may further comprise deriving a diastolic transit time (DTT) value and a plurality of additional properties from the blood pressure waveform. The method may further comprise calculating a calibration factor based on the DTT values and the plurality of additional properties. The method may further comprise calculating estimated DBP values based on the calibration factor, the DTT values, and the plurality of additional properties. The method may further comprise deriving an offset value based on a difference between the one or more estimated DBP values and the one or more raw DBP values, adjusting the blood pressure waveform based on the offset to generate an adjusted blood pressure waveform, and outputting the adjusted blood pressure waveform.

The present invention aimed to overcome the aforementioned shortcomings by developing a new intra-beat biomarker coined as Diastolic Transit Time (DTT) to achieve highly accurate BP estimations. Unlike PTT or PAT which necessitate the use of multi-sensor systems, the algorithm utilizes the slopes of the hemodynamic waveform to enable single-sensor BP monitoring. Compared to other commonly employed signal processing techniques, including bandpass filter (BF), DWT, and STT, the present invention demonstrated superior performance in eliminating stochastic baseline wander, while maintaining signal integrity and BP estimation accuracy in the context of significant hemodynamic changes. This novel algorithm was applied in a demographically and medically diverse cohort of 15 OR patients and showed that the present invention could achieve high correlations between CAP sensor and A-line BP measurements in the context of stress- and drug-induced hemodynamic perturbations for as long as 20 minutes without re-calibration (FIGS. 2A-2B). Furthermore, the present invention's generalizability and ability to be applied to other waveforms was established by demonstrating its efficacy in correlating PPG waveforms obtained from ICU patients to A-line measurements. Using BPV as a spatiotemporal signal measure, the present algorithm was confirmed to not significantly alter BP signal integrity. Moreover, as a proof-of-concept, the present algorithm was demonstrated to be applied to BPV analyses by identifying associations between beat-to-beat BPV and age, hypertension, and vascular disease. Finally, to establish the present invention's potential for future applications in ambulatory or outpatient monitoring, its performance in the context of motion artifacts was shown and demonstrated that, when applied to CAP sensor measurements, the DTT algorithm was able to compensate for baseline shifts from sudden arm/hand movements and walking.

Accurate continuous non-invasive blood pressure (CNIBP) monitoring is the holy grail of digital medicine but remains elusive largely due to significant drifts in signal and motion artifacts that necessitate frequent device recalibration. To address these challenges, the present invention features a unique approach using a novel intra-beat biomarker to achieve highly accurate blood pressure (BP) estimations. The present system demonstrated superior performance compared to other common signal processing techniques, in eliminating stochastic baseline wander, while maintaining signal integrity and measurement accuracy, even during significant hemodynamic changes. The algorithm was applied to a diverse cohort of high acuity patients and demonstrated that it could achieve close agreement between the noninvasive sensor and invasive arterial line BP measurements for up to 20 minutes without recalibration. The present approach's generalizability was established by successfully applying it to pulse waveforms obtained from photoplethysmography and capacitive pressure sensors. The algorithm also maintained signal integrity, enabling reliable assessments of BP variability. Moreover, the algorithm demonstrated tolerance to both low- and high-frequency motion artifacts during abrupt hand movements and prolonged periods of walking. Thus, the present approach shows promise in constituting a necessary advance for wearable sensors for CNIBP monitoring in ambulatory and inpatient settings.

One of the unique and inventive technical features of the present invention is the generation of estimated hemodynamic values through the use of initial and raw hemodynamic values. Without wishing to limit the invention to any theory or mechanism, it is believed that the technical feature of the present invention advantageously provides for single sensor non-invasive blood pressure monitoring that is able to eliminate baseline drift and obviate frequent recalibration. None of the presently known prior references or work has the unique inventive technical feature of the present invention.

Furthermore, the inventive technical feature of the present invention contributed to a surprising result. One skilled in the art would only implement data from a single point on a single waveform to estimate blood pressure values through methods well known in the art. The present invention implements multiple points of data from a plurality of heartbeats to estimate diastolic blood pressure values. Surprisingly, the present invention achieves greater energy- and time-efficiency by analyzing data from a plurality of heartbeats instead of deriving data from only one point on one heartbeat. Thus, the inventive technical feature of the present invention contributed to a surprising result.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a flow chart of the method for continuous, non-invasive, beat-to-beat hemodynamic monitoring of the present invention.

FIG. 1B shows a flow chart of the method for continuous, non-invasive, beat-to-beat blood pressure monitoring of the present invention.

FIG. 2A shows a diagram of the experimental system of the present invention. Inpatient BP data was acquired using an invasive radial A-line and contralaterally-placed noninvasive CAP or PPG sensor.

FIG. 2B shows a graph gathered from an exemplary embodiment of the present invention. BP signals from noninvasive sensors were processed using the DTT algorithm to obtain measurements comparable to those of the A-line. Shaded region indicates the offset between raw (line a) and DTT-processed (line b) signals.

FIG. 3A shows a 300-second BP recording from the CAP sensor was processed using four different methods: bandpass filter (BF), discrete wavelet transformation (DWT), slope transit time (STT), and diastolic transit time (DTT).

FIG. 3B shows BP estimates from the processed signals compared to A-line measurements to assess errors in DBP (line a) and SBP (line b).

FIGS. 4A-4C show a Pearson correlation comparing beat-to-beat SBP, DBP, and MAP measurements respectively between the non-invasive CAP sensor and the invasive A-line sensor. CAP sensor measurements (n=11,002) showed strong linear correlations to the A-line, with Pearson coefficients of 0.987, 0.960, and 0.980 for SBP, DBP, and MAP, respectively.

FIGS. 4D-4F show Bland-Altman analyses comparing beat-to-beat SBP, DBP, and MAP measurements respectively between the non-invasive CAP sensor and the invasive A-line sensor. CAP sensor measurements (n=11,002) demonstrated mean biases of 0.05 (3.07), −0.21 (2.47), and −0.12 (2.35) mmHg for SBP, DBP, and MAP, respectively. Light-and dark-shaded areas represent 68% (1 SD) and 95% (2 SD) limits of agreement, respectively.

FIGS. 5A-5C show a Pearson correlation comparing beat-to-beat SBP, DBP, and MAP measurements respectively between the non-invasive PPG sensor and the invasive A-line sensor. PPG sensor measurements (n=9,628) showed strong linear correlations to the A-line, with Pearson coefficients of 0.982, 0.958, and 0.952 for SBP, DBP, and MAP, respectively.

FIGS. 5D-5F show Bland-Altman analyses comparing beat-to-beat SBP, DBP, and MAP measurements respectively between the non-invasive PPG sensor and the invasive A-line sensor. PPG sensor measurements (n=9,628) demonstrated mean biases of −0.14 (3.20), 0.36 (1.99), and 0.19 (2.09) mmHg for SBP, DBP, and MAP, respectively. Light- and dark-shaded areas represent 68% (1 SD) and 95% (2 SD) limits of agreement, respectively.

FIGS. 6A-6B show stratified graphs comparing SBPV values and DBPV values of patients that have not had a stroke (labeled by a) and that have had a stroke (labeled by b). BPV was assessed using three metrics: standard deviation (SD), coefficient of variation (COV), and average real variability (ARV). * signifies p<0.05.

FIGS. 6C-6D show stratified graphs comparing SBPV values and DBPV values of patients that have not had a stroke (labeled by a) and that have had a stroke (labeled by b) at a very low frequency (nVLF), a low frequency (nLF), and a high frequency (nHF). * signifies p<0.05.

FIGS. 7A-7E show CAP sensor (line a) and Caretaker (arrows labeled by b) BP measurements with corresponding normalized accelerometer signal (line c) during various exercises. FIG. 7A shows the aforementioned measurements during a 180° wrist rotation. FIG. 7B shows the aforementioned measurements during a 90° wrist flexion. FIG. 7C shows the aforementioned measurements during hand closure. FIG. 7D shows the aforementioned measurements during a wrist hit/impulse. FIG. 7E shows the aforementioned measurements during walking. Shaded regions indicate periods of movement. Upward and downward facing arrows represent Caretaker SBP and DBP, respectively.

FIG. 8 shows a table of surgical patient demographics.

FIG. 9 shows a table depicting measurements of ARV, COV, and SD for age-, hypertension-, and vascular disease-stratified surgical patients.

### DETAILED DESCRIPTION OF THE INVENTION

Following is a list of elements corresponding to a particular element referred to herein:

100 sensor

200 computing device

The present invention features a system for continuous, non-invasive, beat-to-beat hemodynamic monitoring of a subject. In some embodiments, the system may comprise a sensor (100) coupled to the subject. The sensor (100) may be capable of measuring a hemodynamic waveform comprising a plurality of heartbeats based on an unadjusted hemodynamic signal. The system may further comprise a computing device (200) communicatively coupled to the sensor (100). The computing device (200) may comprise a processor capable of executing computer-readable instructions, and a memory component comprising computer-readable instructions. The computer-readable instructions may comprise receiving the hemodynamic waveform from the sensor (100) and deriving an initial systolic hemodynamic value and an initial waveform contractility value from the hemodynamic waveform. The computer-readable instructions may further comprise deriving a diastolic transit time (DTT) value, a pulse pressure (PP) value, a raw diastolic hemodynamic value, a systolic hemodynamic value, and a waveform contractility value from the hemodynamic waveform for one or more heartbeats of the plurality of heartbeats.

The computer-readable instructions may further comprise calculating a calibration factor based on one or more of the DTT values, one or more of the PP values, one or more of the raw diastolic hemodynamic values, and one or more of the systolic hemodynamic values over one or more heartbeats of the plurality of heartbeats. In some embodiments, this may be over the first five heartbeats of the unadjusted signal. In other embodiments, this may be over 3 to 10 initial heartbeats of the unadjusted signal. In other embodiments, this may be over any number of heartbeats of the unadjusted signal. The computer-readable instructions may further comprise calculating one or more estimated diastolic hemodynamic values based on the calibration factor, the initial systolic hemodynamic value, the initial waveform contractility value, one or more of the DTT values, and one or more of the waveform contractility values over one or more heartbeats of the plurality of heartbeats. The computer-readable instructions may further comprise deriving an offset value based on a difference between the one or more estimated diastolic hemodynamic values and the one or more raw diastolic hemodynamic values, adjusting the hemodynamic waveform based on the offset to generate an adjusted hemodynamic waveform, and outputting the adjusted hemodynamic waveform.

In some embodiments, the sensor (100) may comprise a capacitive pressure sensor, a photoplethysmograph sensor, a speckleplethysmograph sensor, an optical sensor, a tonometry-based device, or a combination thereof. The sensor (100) may be communicatively coupled to the computing device (200) by a wireless component or a wired component. In some embodiments, the hemodynamic waveform, the systolic hemodynamic value(s), the diastolic hemodynamic value(s), or a combination thereof may comprise information on blood pressure, cardiac output, vascular elasticity, and autonomic function. The waveform contractility value may comprise a slope of the systolic upstroke of the waveform.

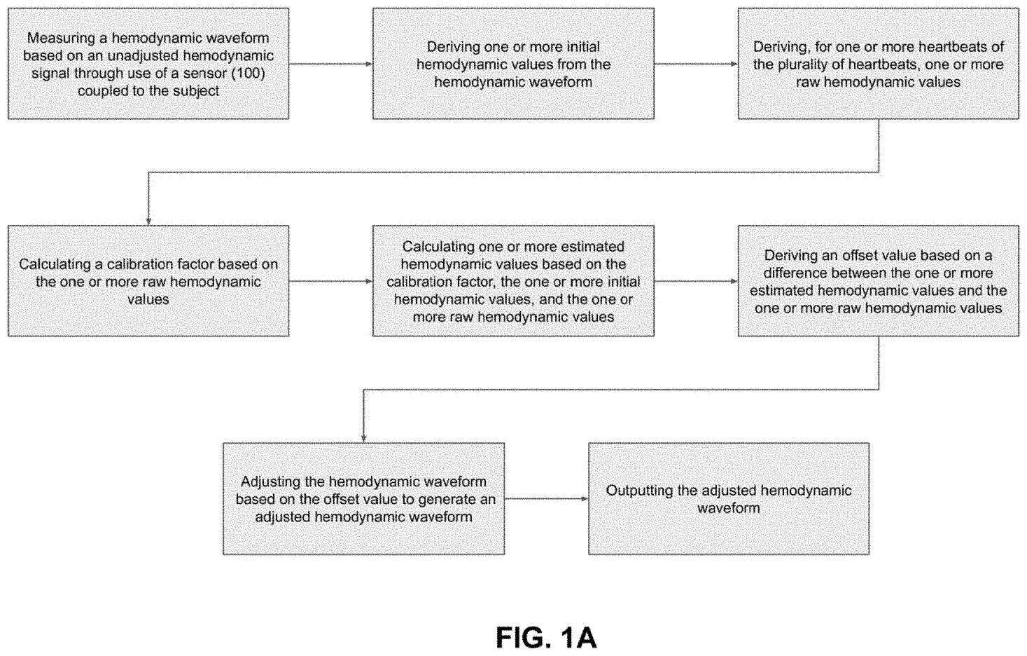

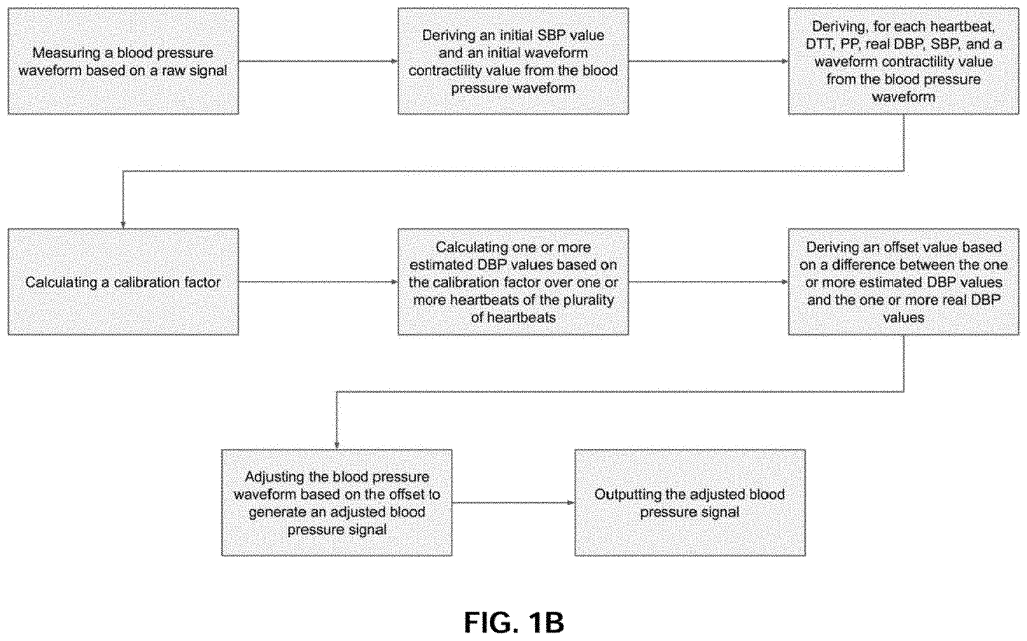

Referring now to FIG. 1, the present invention features a method for continuous, non-invasive, beat-to-beat hemodynamic monitoring of a subject. In some embodiments, the method may comprise measuring a hemodynamic waveform based on an unadjusted hemodynamic signal through the use of a sensor (100) coupled to the subject. The unadjusted hemodynamic signal may comprise a plurality of heartbeats. The method may further comprise deriving an initial systolic hemodynamic value and an initial waveform contractility value from the hemodynamic waveform, and deriving a diastolic transit time (DTT) value, a pulse pressure (PP) value, a raw diastolic hemodynamic value, a systolic hemodynamic value, and a waveform contractility value from the hemodynamic waveform for one or more heartbeats of the plurality of heartbeats.

The method may further comprise calculating a calibration factor based on one or more of the DTT values, one or more of the PP values, one or more of the raw diastolic hemodynamic values, and one or more of the systolic hemodynamic values over one or more heartbeats of the plurality of heartbeats. The method may further comprise calculating one or more estimated diastolic hemodynamic values based on the calibration factor, the initial systolic hemodynamic value, the initial waveform contractility value, one or more of the DTT values, and one or more of the waveform contractility values over one or more heartbeats of the plurality of heartbeats. The method may further comprise deriving an offset value based on a difference between the one or more estimated diastolic hemodynamic values and the one or more raw diastolic hemodynamic values, adjusting the hemodynamic waveform based on the offset to generate an adjusted hemodynamic waveform, and outputting the adjusted hemodynamic waveform. In some embodiments, the sensor (100) may comprise a capacitive pressure sensor, a photoplethysmograph sensor, a speckleplethysmograph sensor, an optical sensor, a tonometry-based device, or a combination thereof. The sensor (100) may be communicatively coupled to a computing device (200) by a wireless component or a wired component. The unadjusted hemodynamic signal may be representative of information on blood pressure, cardiac output, vascular elasticity, and autonomic function. In some embodiments, the hemodynamic waveform, the systolic hemodynamic value(s), the diastolic hemodynamic value(s), or a combination thereof may comprise information on blood pressure, cardiac output, vascular elasticity, and autonomic function. The waveform contractility value may comprise a slope of the systolic upstroke of the waveform.

The present invention features a method for continuous, non-invasive, beat-to-beat blood pressure monitoring of a subject. In some embodiments, the method may comprise measuring a blood pressure waveform based on an unadjusted hemodynamic signal through the use of a sensor (100) coupled to the subject. The unadjusted hemodynamic signal may comprise a plurality of heartbeats. The method may further comprise deriving an initial systolic hemodynamic value and an initial waveform contractility value from the blood pressure waveform, and deriving a diastolic transit time (DTT) value, a pulse pressure (PP) value, a raw diastolic blood pressure (DBP) value, a systolic hemodynamic value, and a waveform contractility value from the blood pressure waveform for one or more heartbeats of the plurality of heartbeats.

The method may further comprise calculating, for one or more heartbeats of the plurality of heartbeats, one or more estimated DBP (eDBP) values using the following predefined formula:

$$eDBP ( t ) = {SBP}_{0} - [ {m}_{0} * DTT ( t ) * {( \frac{C ( t )}{{C}_{0}} )}^{- 1} ] , \qquad ( Equation 1 )$$

wherein t=time, SBP=systolic blood pressure, SBP₀=initial systolic blood pressure (e.g., initial SBP cuff measurement), DTT=diastolic transit time, C=waveform contractility, C₀=initial waveform contractility, and m₀ is a calibration factor calculated as an average of the first b recorded beats and defined by the following formula:

$${m}_{0} = \frac{1}{b} * {\sum }_{i = 1}^{b} \frac{{PP}_{0}}{{PP}_{s} ( i )} * \frac{{SBP}_{s} ( i ) - {DBP}_{s} ( i + 1 )}{DTT ( i )} , \qquad ( Equation 2 )$$

wherein b=heartbeats, PP₀=initial pulse pressure (e.g., initial pulse pressure obtained by cuff measurement), PPₛ=sensor pulse pressure, SBPₛ was the sensor SBP, DBP=raw diastolic blood pressure, and DBPₛ was the sensor DBP.

In some embodiments, the method may further comprise deriving an offset value based on a difference between the one or more estimated DBP values and the one or more raw DBP values, adjusting the blood pressure waveform based on the offset to generate an adjusted blood pressure waveform, and outputting the adjusted blood pressure waveform.

In some embodiments, the sensor (100) may comprise a capacitive pressure sensor, a photoplethysmograph sensor, a speckleplethysmograph sensor, an optical sensor, a tonometry-based device, or a combination thereof. The sensor (100) may be communicatively coupled to a computing device (200) by a wireless component or a wired component.

The present invention features a system for continuous, non-invasive, beat-to-beat hemodynamic monitoring of a subject. In some embodiments, the system may comprise a sensor (100) coupled to the subject. The sensor (100) may be configured to measure a hemodynamic waveform comprising a plurality of heartbeats based on an unadjusted hemodynamic signal. The system may further comprise a computing device (200) communicatively coupled to the sensor (100), comprising a processor configured to execute computer-readable instructions, and a memory component comprising computer-readable instructions. The computer-readable instructions may comprise receiving the hemodynamic waveform from the sensor (100), deriving one or more initial hemodynamic values from the hemodynamic waveform, deriving, for one or more heartbeats of the plurality of heartbeats, one or more raw hemodynamic values, calculating a calibration factor based on the one or more raw hemodynamic values, calculating one or more estimated hemodynamic values based on the calibration factor, the one or more initial hemodynamic values, and the one or more raw hemodynamic values, deriving an offset value based on a difference between the one or more estimated hemodynamic values and the one or more raw hemodynamic values, adjusting the hemodynamic waveform based on the offset value to generate an adjusted hemodynamic waveform, and outputting the adjusted hemodynamic waveform.

The present invention features a method for continuous, non-invasive, beat-to-beat hemodynamic monitoring of a subject. In some embodiments, the method may comprise measuring a hemodynamic waveform based on an unadjusted hemodynamic signal through use of a sensor (100) coupled to the subject. The unadjusted hemodynamic signal may comprises a plurality of heartbeats. The method may further comprise deriving one or more initial hemodynamic values from the hemodynamic waveform, deriving, for one or more heartbeats of the plurality of heartbeats, one or more raw hemodynamic values, calculating a calibration factor based on the one or more raw hemodynamic values, calculating one or more estimated hemodynamic values based on the calibration factor, the one or more initial hemodynamic values, and the one or more raw hemodynamic values, deriving an offset value based on a difference between the one or more estimated hemodynamic values and the one or more raw hemodynamic values, adjusting the hemodynamic waveform based on the offset value to generate an adjusted hemodynamic waveform, and outputting the adjusted hemodynamic waveform.

The one or more initial hemodynamic values may comprise an initial systolic hemodynamic value and an initial waveform contractility value derived from the hemodynamic waveform. The one or more raw hemodynamic values may comprise a diastolic transit time (DTT) value, a pulse pressure (PP) value, a raw diastolic hemodynamic value, a systolic hemodynamic value, and a waveform contractility value derived from the hemodynamic waveform. The method of claim 6, wherein the system is configured for continuous, non-invasive, beat-to-beat hemodynamic monitoring of a subject through the use of only one sensor (100). The system of the present invention may be configured for continuous, non-invasive, beat-to-beat hemodynamic monitoring of a subject through the use of only one sensor (100).

Using an initial BP measurement from a standard BP cuff, beat-to-beat diastolic blood pressure (DBP) was calculated using Equation 1 and Equation 2 as a function of DTT, defined as the time from systolic peak to diastolic trough, and waveform contractility. Thus, the calibration factor, m₀ [mm Hg/s], served two purposes: (1) to convert the raw signal into pressure measurements [mm Hg] and (2) to track changes in DBP relative to the initial DTT. Beat-to-beat change in contractility was included as a dynamic transformation factor to adjust for stress- or drug-induced physiological changes in left ventricular contractility that can modify contraction and relaxation times and, consequently, alter BP waveform morphology.

Since the algorithm calculated DBP using intra-beat parameters that were independent of the confounding effects introduced by low-frequency noise, by comparing the estimated DBP to raw DBP, the baseline wander was empirically modeled in recordings, which served as an offset for the raw BP signals. The calculated baseline wander was smoothed using a 30-point moving median filter before being subtracted from the raw BP signal. The corrected signal was then used to extract beat-to-beat DBP, SBP, and mean arterial pressure (MAP, Equation 3). Outlier measurements were excluded using a 30-point moving median filter.

$$MAP = DBP + \frac{1}{3} * ( SBP - DBP ) \qquad ( Equation 3 )$$

Unlike PTT or PAT that necessitate the use of multi-sensor systems, the DTT algorithm utilizes the slopes of the hemodynamic waveform to enable single-sensor BP monitoring. Compared to other commonly employed signal processing techniques, including bandpass filter (BF), DWT, and STT, the approach's superior performance in eliminating stochastic baseline wander was demonstrated, while maintaining signal integrity and BP estimation accuracy in the context of significant hemodynamic changes. This novel algorithm was applied in a demographically and medically diverse cohort of 15 OR patients and showed that it could achieve high correlations between sensor and A-line BP measurements in the context of stress- and drug-induced hemodynamic perturbations for as long as 20 minutes without re-calibration. Furthermore, this established the approach's generalizability and ability to be applied to other waveforms by demonstrating its efficacy in correlating PPG waveforms obtained from ICU patients to A-line measurements.

### EXAMPLE

The following is a non-limiting example of the present invention. It is to be understood that said example is not intended to limit the present invention in any way. Equivalents or substitutes are within the scope of the present invention.

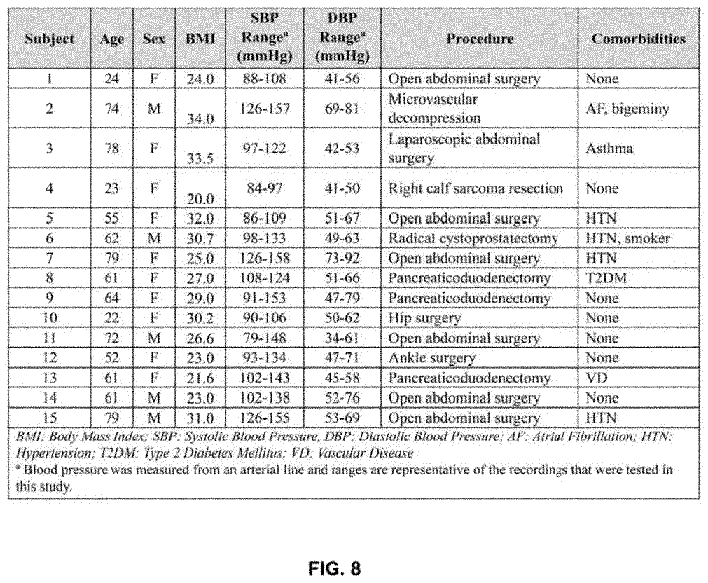

BP data was obtained from 15 surgical patients (FIG. 8), 10 of whom were female. For the OR cohort, the average age was 57.8 (range: 22-79) years and the average BMI was 27.4 (range: 20.0-34.0) kg/m². A total of 10,226 seconds of intra-operative BP recordings were extracted, with an average segment length of 204.5 (range: 60-1200) seconds. The ICU cohort consisted of 20 BP recordings, totaling 8,405 seconds in duration, from different patients being treated at an ICU. The average segment length of the ICU recordings was 420.3 (range: 180-590) seconds. Due to the anonymized nature of the database, demographics were not available for the ICU patients.

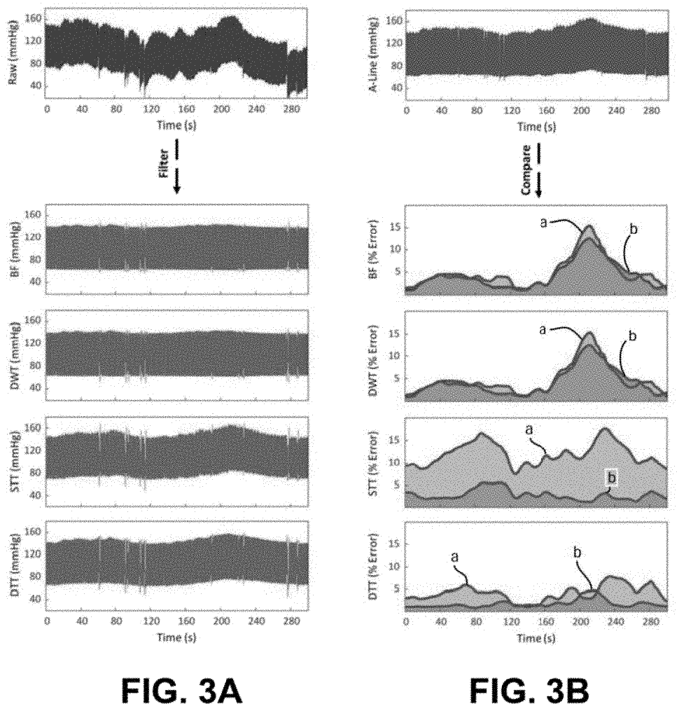

To compare the algorithm's accuracy, calibration dependency, and BP tracking ability with those of other commonly employed methods, a 300-second segment of CAP sensor and A-line BP recordings from a hemodynamically unstable surgical patient was utilized (FIGS. 3A-3B). The algorithm was compared to 3 common methods: bandpass filtering (BF) using a 4^{th} order Chebyshev II filter with cutoff frequencies of 0.5 and 10 Hz reduction of 7^{th} level approximation coefficients using DWT with Daubechies 4 wavelets and BP estimation using the STT approach. When applied to the raw BP signal, the DTT algorithm demonstrated average SBP and DBP errors of 1.69%±0.85 and 4.15%±1.70, respectively. In contrast, average SBP errors were found to be significantly higher using the BF (4.48%±3.07), DWT (4.70%±3.51), and STT (2.80%±1.10) algorithms (all p<0.001). Moreover, average DBP errors were higher using the DWT (4.38%±3.06, p=0.048) and STT (11.98%±2.50, p<0.001) methods. While measurement errors from the DTT algorithm were consistently below 8%, significant deviations in accuracy were observed with the other algorithms during hemodynamic changes (e.g., increasing BP), with errors surpassing 15%.

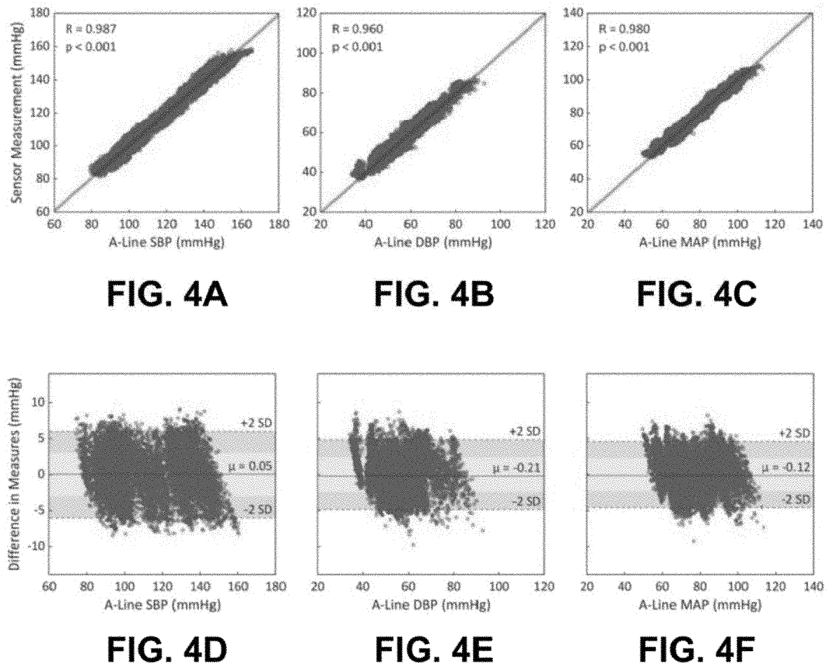

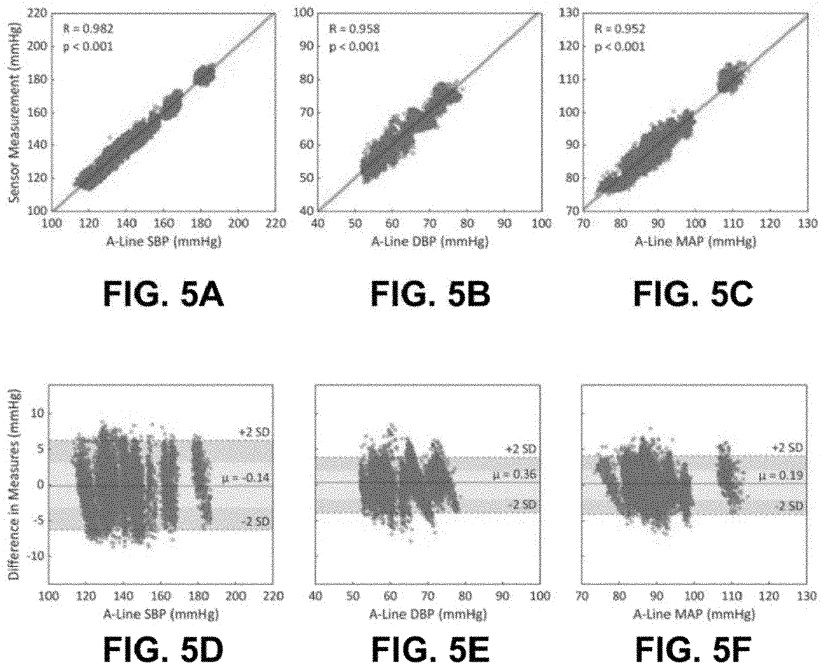

Using BP recordings from high acuity patients (OR and ICU), the algorithm's ability to correct raw BP signals and accurately measure critical cardiovascular parameters was evaluated (FIGS. 4A-5F). Overall, the algorithm was applied across a wide hemodynamic range: SBP 80-184 mmHg, DBP 34-92 mmHg, MAP 49-115 mmHg, HR 44-123 beats per minute (bpm).

The OR cohort demonstrated strong linear correlations between CAP sensor estimations and gold standard A-line measurements, with Pearson coefficients of 0.987, 0.960, and 0.980 for SBP, DBP, and MAP, respectively (FIGS. 4A-4C). The resulting mean bias (SD) for SBP, DBP, and MAP were 0.05 (3.07), −0.21 (2.47), and −0.12 (2.35) mmHg, respectively (FIGS. 4D-4F). Additionally, HR measurements from the CAP sensor strongly agreed with those from the A-line (mean bias: 0.02±1.53 bpm). This was a stark improvement from measurements obtained without using the DTT algorithm, which exhibited mean biases (SD) of −6.63 (16.30), −6.56 (16.35), and −6.59 (16.20) for SBP, DBP, and MAP, respectively.

To demonstrate the generalizability of the DTT algorithm to other modalities, the accuracy of the approach was further evaluated by applying it to PPG measurements obtained from ICU patients. The present algorithm similarly showed strong linear correlations to the A-line, with Pearson coefficients of 0.982, 0.958, and 0.952 for SBP, DBP, and MAP, respectively (FIGS. 5A-5C). Moreover, the present algorithm demonstrated high estimation accuracies, with mean bias (SD) of −0.14 (3.20), 0.36 (1.99), and 0.19 (2.09) mmHg for SBP, DBP, and MAP, respectively (FIGS. 5D-5F). Additionally, HR measurements from the PPG sensor strongly agreed with those from the A-line (mean bias: −0.02±1.58 bpm). This was once again a significant improvement in accuracy compared to measurements obtained without using the DTT algorithm, which exhibited mean biases (SD) of −1.71 (7.20), −1.12 (5.20), and −1.32 (5.08) for SBP, DBP, and MAP, respectively.

Assessment of beat-to-beat BPV can provide unique perspectives on important cardiovascular parameters and physiologic states. Thus, to verify that the present approach maintained BPV integrity, the DTT algorithm was applied to the gold standard A-line measurements for all 15 OR patients and evaluated if BPV would be significantly altered. On average, the SDs of processed and unprocessed A-line measurements were 1.35 and 1.40 mmHg for SBP, 0.97 and 0.90 mmHg for DBP, and 1.02 and 1.01 mmHg for MAP, respectively. Overall, there was no statistically significant difference in BPV between these two groups for SBP (p=0.351), DBP (p=0.272), and MAP (p=0.426).

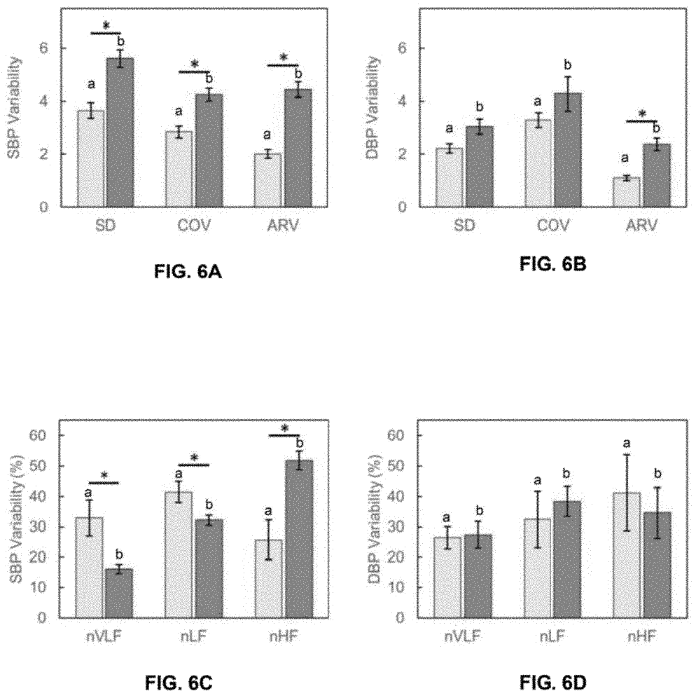

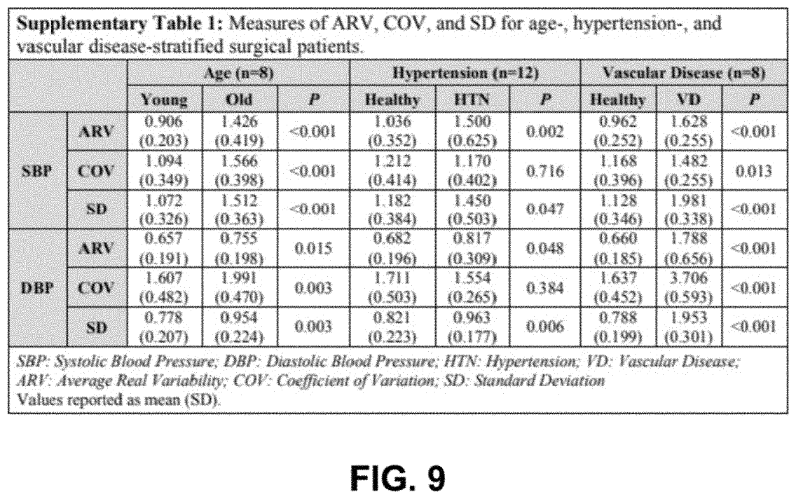

Hence, to uncover associations between BPV and cardiovascular health, OR patients were stratified into cohorts according to their age, history of hypertension, and history of vascular disease (VD). The systolic (SBPV) and diastolic BPV (DBPV) of subjects were subsequently evaluated and compared across groups (FIGS. 6A-6F). In the age-stratified cohort, older subjects (>60 years old) were found to have significantly higher SBPV and DBPV than younger subjects (<30 years old) across all variability indices (all p<0.05, FIG. 9). On the other hand, hypertensive patients demonstrated significantly higher SD and average real variability (ARV) than healthy patients for both SBP and DBP (all p<0.05). Finally, vascular disease was found to be associated with a significantly higher SBPV and DBPV (all p<0.05).

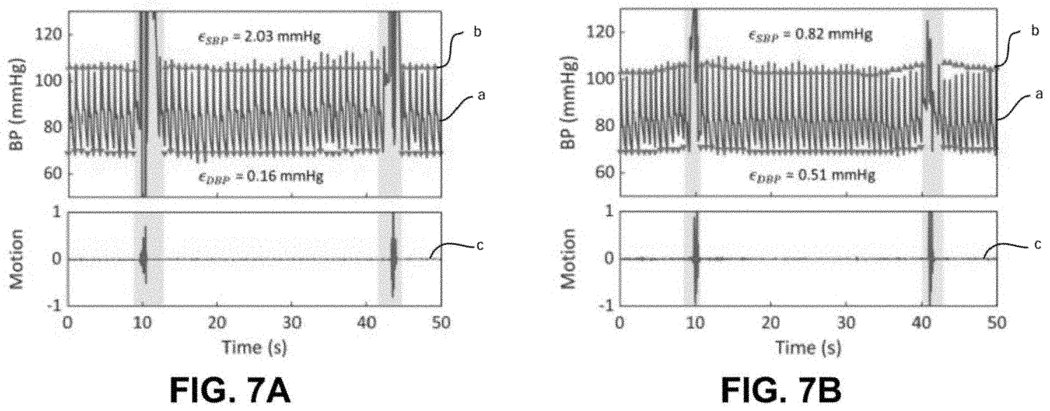

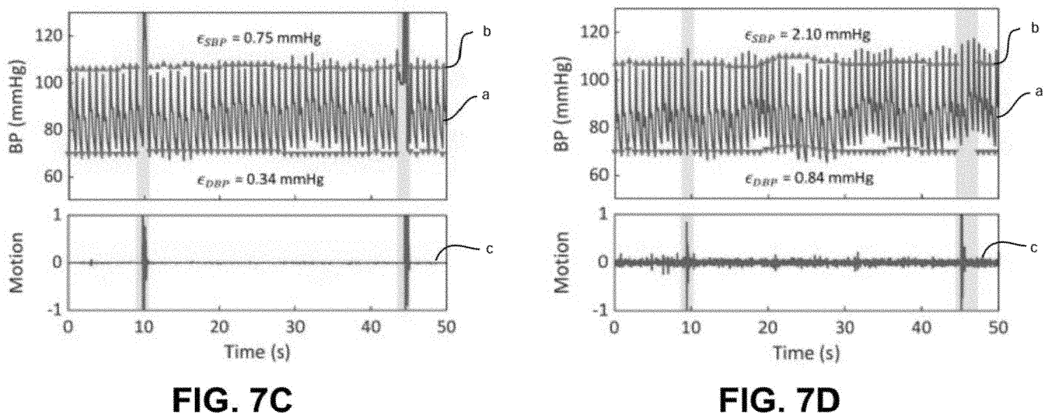

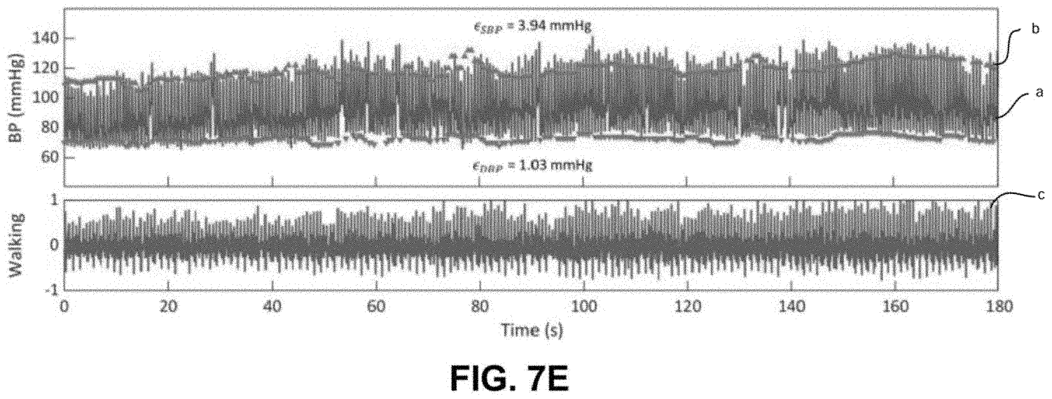

Intermittent motion artifacts present a significant challenge for ambulatory and outpatient CNIBP. To investigate the algorithm's potential for future implementation into these settings, its performance in correcting CAP sensor measurements was assessed in the context of common arm/hand movements—180° wrist rotation, 90° wrist flexion, hand closure, and wrist hit/impulse—and walking (FIGS. 7A-7E). Movements were tracked using an accelerometer embedded in the CAP sensor's wireless board, which was attached to the subject's hand using an elastic strap. Motion was represented by the normalized magnitude (−1 to 1) of the accelerometer's measurements.

Overall, the processed CAP sensor signals exhibited excellent agreement with Caretaker measurements. On average, the mean biases (SD) were 2.03 (2.55), 0.82 (2.83), 0.75 (2.6), and 2.10 (3.60) mmHg for SBP and 0.16 (2.09), 0.51 (1.86), 0.34 (2.16), and 0.84 (3.73) mmHg for DBP during wrist rotation, wrist flexion, hand closure, and wrist hit/impulse, respectively. Additionally, over the duration of three minutes of walking, the algorithm's BP estimates agreed well with Caretaker measurements and demonstrated mean biases of 3.94 (5.49) and 1.03 (3.23) mmHg for SBP and DBP, respectively.

In this study, the efficacy of the novel DTT approach in eliminating stochastic baseline wander to achieve accurate beat-to-beat BP measurements was demonstrated, well within the limits demanded by AAMI/ISO standards, using noninvasive CAP and PPG sensor recordings from surgical and ICU patients. Despite their many advancements over the years, CNIBP monitors (e.g., PPG, tonometry) all continue to face significant baseline drift and noise that prevent accurate, long-term continuous BP measurements. For example, Kaisti et al. (Kaisti, M. et al. Clinical assessment of a non-invasive wearable MEMS pressure sensor array for monitoring of arterial pulse waveform, heart rate and detection of atrial fibrillation. npj Digit. Med. 2, 39 (2019)) recently developed a non-invasive wearable MEMS pressure sensor that demonstrated remarkable temporal and morphological BP waveform accuracy; however, they were unable to measure BP amplitude due to reported baseline variations. Some devices have attempted to circumvent this issue by requiring recalibration as frequently as every minute. Naturally, this has made such devices often impractical and has stymied their adoption into clinical practice. Therefore, the algorithm's ability to effectively remove this problematic drift and overcome this long-existing challenge can have significant implications in bringing existing and future CNIBP monitoring technologies closer to clinical use. Additionally, unlike many methods that require a posteriori signal processing, such as filters or regressions, or machine learning-based approaches that are computationally expensive and require training on large datasets, the DTT approach is algorithmically simple and may be more adaptable to real-time monitoring applications.

Since a number of cardiovascular changes occur at varying BPs and heart rates, measurement techniques need to be validated across a wide hemodynamic range before being considered sufficiently reliable for use in clinical practice. Thus, by using a heterogeneous and hemodynamically labile cohort of patients for the analyses, the algorithm's performance across a wide range of physiologic states could be evaluated to establish its validity for clinical use. However, nearly 52% of prior studies have reportedly used only normotensive patients for testing, severely limiting their external validity. Moreover, only 4% of studies have included hypotensive patients and fewer than 8% have included hypertensive patients in their analyses. Therefore, this study serves as one of a select series of reports that have investigated CNIBP monitoring accuracy across a representative cohort of patients.

The ability for an algorithm to accurately track BP changes is also an important dynamic measure that is rarely assessed. Studies have frequently utilized pre-processing methods, such as BF and DWT, to reduce noise and baseline drift, with seemingly acceptable results. However, since these methods are commonly applied to healthy, young subjects and their results reported as aggregate averages, it is often not possible to characterize their behaviors in the context of active BP changes. By utilizing a hemodynamically labile segment of BP measurements, the approach's ability to track BP changes was calculated and compared with other common techniques. As hypothesized, the accuracies of BF- and DWT-based methods faltered during changes in BP. This is likely due to the filters' inability to distinguish between artificial (i.e., noise) and physiological BP drift since the basis of both methods relies on signal frequency without considering individual waveform characteristics. On the other hand, STT-based BP estimation exhibited slightly higher errors in SBP, but significantly greater deviations in DBP than the approach. As the BP waveform can be affected by many factors, it is possible that the STT method's reliance on a single systolic feature limited its ability to reflect changes in both SBP and DBP.

Validation experiments indicated that the DTT algorithm did not significantly alter BP signal variability, suggesting that the present approach may be applied to future studies on short-term BPV. The experiments supported this conclusion, as these were able to identify several interesting associations between short-term BPV and cardiovascular health. These findings demonstrated that older age was associated with increased SBPV and DBPV, in agreement with other studies that have suggested it to be due to increased arterial stiffness and impaired baroreceptor function. Additionally, patients with hypertension exhibited higher ARVs and SDs for SBP and DBP than healthy individuals. This is consistent with Xia et. al's findings, which suggested that hypertensive patients may possess compromised vascular elasticity, and hence elevated BPV. While there was not a significant difference in coefficient of variation (COV) between the two groups, this may have been attributed to a type II error from a limited sample size or due to COV's lower sensitivity for short-term changes than ARV. Finally, the experiments demonstrated a significantly higher SBPV and DBPV in vascular disease patients than healthy individuals. Interestingly, there was an observably larger inter-group difference in DBPV than SBPV. This was likely a result of the characteristic increase in arterial wall stiffness associated with vascular disease.

As the demand increases for wearable devices that enable continuous day-to-day biomonitoring, significant efforts have been made towards developing systems that support long-term ambulatory recording. However, despite many advancements in CNIBP monitoring technologies over the years, ambulatory BP monitoring continues to be an elusive undertaking. The presence of motion-related artifacts and abrupt changes in signal baseline introduce an especially complex confounding factor in BP estimation algorithms that make accurate and precise ambulatory BP monitoring a significant challenge. While several promising techniques have been recently developed, corn pensating for motion artifacts, especially those from—macro-motionsll during walking or jogging, that possess an overlapping frequency spectrum with the BP signal remains a challenge. In these experiments, it was shown that the DTT algorithm was able to recover quickly from sudden baseline shifts caused by abrupt hand/arm movements. Importantly, this demonstrated the algorithm's high tolerance to low- (e.g., arm swing) and high-frequency (e.g., step impulse) motion artifacts through its ability to accurately measure beat-to-beat BP during a prolonged period of walking. While additional testing during more intense activities is warranted, the DTT approach, nonetheless, shows potential as a means towards bringing CNIBP monitoring to the ambulatory setting.

Although the present approach overcomes several critical obstacles in CNIBP monitoring, there are still limitations that warrant future study. While the DTT algorithm mitigates much of the challenges associated with baseline wander and motion artifacts, as with existing CNIBP technologies, it is still susceptible to the confounding effects incurred by changing applanation pressure. Quantitative characterization of pulse waveforms shows promise as a method of automatically assessing signal quality and detecting perturbations in applanation. Several groups have also demonstrated the feasibility of using a secondary pressure sensor or force transducer as a feedback mechanism within their system to account for changes in contact pressure. To demonstrate the DTT approach, segments of data with visibly low-quality waveforms were excluded, and a moving median filter was utilized as a coarse method to remove remaining intermittent disruptions in signal quality. Future iterations of the algorithm may benefit from a machine learning classification model (e.g., support-vector machine, logistic regression) to automatically assess signal quality and more precisely exclude corrupt waveforms.

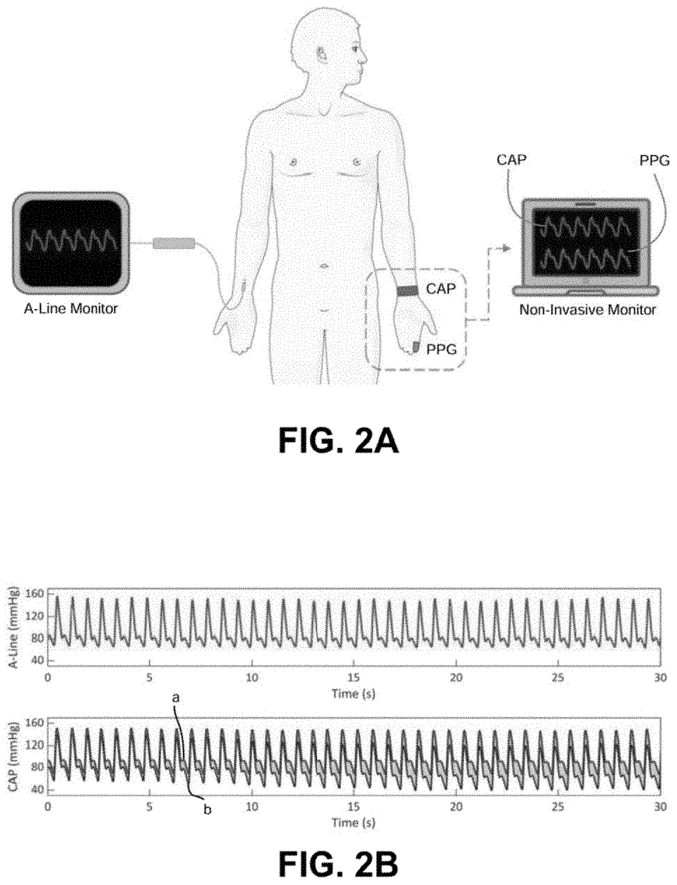

BP data was acquired from subjects in the OR, ICU, and ambulatory settings. The OR cohort consisted of 15 intraoperative patients receiving treatment at the University of California, Irvine (UCI) Medical Center between June 2020 and March 2021. All patients were under general anesthesia and received intravenous medications (e.g., ephedrine, phenylephrine) whenever clinically indicated. BP recordings from surgical patients were obtained invasively via radial A-line and noninvasively using a CAP sensor placed on the contralateral radial artery (FIG. 2A). BP data for the ICU cohort was obtained from a public database (UCI Machine Learning Repository) and consisted of 20 randomly-selected recordings of invasive radial A-line and non-invasive radial PPG measurements collected from patients being treated at ICU facilities. Ambulatory BP measurements used for motion artifact analysis were obtained from one healthy subject using an FDA-cleared CNIBP monitoring device (Caretaker®; Caretaker Medical NA, Charlottesville, VA, USA) and a noninvasive CAP sensor placed at the contralateral radial artery. Informed consent was obtained from all OR and ambulatory subjects, and data was acquired in accordance with the UCI Institutional Review Board (IRB no. 2019-5251 and 2016-2924). Usage of the ICU recordings was IRB exempt due to the anonymized and deidentified nature of the public database.

All collected BP signals were pre-processed in MATLAB (R2021a, The MathWorks®, Natick, Massachusetts, USA) prior to analysis. Using the devices' integrated clocks, noninvasive BP measurements (via CAP or PPG sensors) were synchronized with the recordings from reference devices (A-line or Caretaker) to a precision of one second. Next, BP recordings were visually screened by the authors to exclude sections of data with the low-quality signal. Signals were considered of sufficient quality if they consisted of at least 30 seconds of continuous data, in which the BP waveforms possessed clearly visible systolic peaks, dicrotic notches, and diastolic troughs. Since inconsistent applanation pressure is a major source of measurement error in CNIBP monitors, and manual manipulation (e.g., repositioning) of sensors could not be controlled for in the OR and ICU setting, an unsupervised algorithm was developed to objectively identify and exclude segments of data that contained significant deviations in applanation. Since perturbations in contact pressure alter signal amplitude, a change in BP waveform contractility, defined as the maximum of the first derivative of the systolic upstroke, was used as a surrogate marker of changing applanation. Hence, for a given pair of noninvasive and invasive BP signals, regressions of the change in normalized contractility were calculated and compared using a hypothesis test. The sensors were considered to have significant deviations in applanation if the pair of regressions were statistically different (p<0.05). A 30-second sliding window was utilized in this step to minimize the amount of excluded data.

Since there is currently no well-established standard for measuring BPV, short-term (beat-to-beat) SBPV and DBPV were quantified using three different metrics: SD, COV, and ARV. SD is the most commonly used index and represents the global fluctuation of BP measurements around the mean. COV is a normalized measure of SD and is defined by dividing by the mean BP. ARV, which aims to account for the temporal order of measurements and reduce the errors produced by signal noise, was defined by the mean of the absolute differences between adjacent BP measurements. Each BPV measurement represented an average over a 30-beat window. Since intraoperative infusion of vasoactive medications could artificially increase BPV, this confounding factor was mitigated by excluding BPV measurements from segments that exhibited BP ranges exceeding 10 mmHg.

All statistical analyses were performed in MATLAB. A p-value less than 0.05 was considered statistically significant. A t-test or Wilcoxon signed rank test was used for continuous variables to evaluate differences between the means of two samples. Shapiro-Wilk tests were used to assess for normality. Brown-Forsythe tests were used to determine statistical differences in BPV between two sets of measurements. Pearson linear correlation coefficients were calculated to assess how well beat-to-beat noninvasive sensor measurements correlated with those of the A-line. Mean bias, SD, and 95% confidence intervals (CIs) were also calculated, which in combination with the Bland-Altman method of paired measurements, were used to assess agreement between noninvasive (CAP or PPG sensor) and invasive (A-line) BP monitoring methods. The benchmark for acceptance was based on AAMI/ISO 81060-2 standards (mean bias: 5±8 mmHg), which are used for FDA clearance of non-invasive sphygmomanometers.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft® Corporation; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of media. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system for continuous, non-invasive, beat-to-beat hemodynamic monitoring of a subject, the system comprising:
a. a sensor (100) coupled to the subject, wherein the sensor (100 ) is configured to measure a hemodynamic waveform comprising a plurality of heartbeats based on an unadjusted hemodynamic signal; and
b. a computing device (200) communicatively coupled to the sensor (100), comprising a processor configured to execute computer-readable instructions, and a memory component comprising computer-readable instructions for:
i. receiving the hemodynamic waveform from the sensor (100);
ii. deriving one or more initial hemodynamic values from the hemodynamic waveform;
iii. deriving, for one or more heartbeats of the plurality of heartbeats, one or more raw hemodynamic values;
iv. calculating a calibration factor based on the one or more raw hemodynamic values;
v. calculating one or more estimated hemodynamic values based on the calibration factor, the one or more initial hemodynamic values, and the one or more raw hemodynamic values;
vi. deriving an offset value based on a difference between the one or more estimated hemodynamic values and the one or more raw hemodynamic values;
vii. adjusting the hemodynamic waveform based on the offset value to generate an adjusted hemodynamic waveform; and
viii. outputting the adjusted hemodynamic waveform.

2. The system of claim 1, wherein the calibration factor is calculated over 3 to 10 initial heartbeats of the plurality of heartbeats.

3. The system of claim 1, wherein the sensor (100) comprises a capacitive pressure sensor, a photoplethysmograph sensor, speckleplethysmograph sensor, an optical sensor, a tonometry-based device, or a combination thereof.

4. The system of claim 1, wherein the sensor (100) is communicatively coupled to the computing device (200) by a wireless component or a wired component.

5. The system of claim 1, wherein the system is configured for continuous, non-invasive, beat-to-beat hemodynamic monitoring of a subject through the use of only one sensor (100).

6. The system of claim 1, wherein the unadjusted hemodynamic signal is representative of information on blood pressure, cardiac output, vascular elasticity, and autonomic function.

7. A method for continuous, non-invasive, beat-to-beat hemodynamic monitoring of a subject, the method comprising:
a. measuring a hemodynamic waveform based on an unadjusted hemodynamic signal through use of a sensor (100) coupled to the subject, wherein the unadjusted hemodynamic signal comprises a plurality of heartbeats;
b. deriving one or more initial hemodynamic values from the hemodynamic waveform;
c. deriving, for one or more heartbeats of the plurality of heartbeats, one or more raw hemodynamic values;
d. calculating a calibration factor based on the one or more raw hemodynamic values;
e. calculating one or more estimated hemodynamic values based on the calibration factor, the one or more initial hemodynamic values, and the one or more raw hemodynamic values;
f. deriving an offset value based on a difference between the one or more estimated hemodynamic values and the one or more raw hemodynamic values;
g. adjusting the hemodynamic waveform based on the offset value to generate an adjusted hemodynamic waveform; and
h. outputting the adjusted hemodynamic waveform.

8. The method of claim 7, wherein the calibration factor is calculated over 3 to 10 initial heartbeats of the plurality of heartbeats.

9. The method of claim 7, wherein the sensor (100) comprises a capacitive pressure sensor, a photoplethysmograph sensor, speckleplethysmograph sensor, an optical sensor, a tonometry-based device, or a combination thereof.

10. The method of claim 7, wherein the sensor (100) is communicatively coupled to a computing device (200).

11. The method of claim 10, wherein the sensor (100) is communicatively coupled to the computing device (200) by a wireless component or a wired component.

12. The method of claim 7, wherein measuring a hemodynamic waveform based on an unadjusted hemodynamic signal through use of a sensor (100) comprises measuring through the use of only one sensor (100).

13. The method of claim 7, wherein the unadjusted hemodynamic signal is representative of information on blood pressure, cardiac output, vascular elasticity, and autonomic function.

14. A method for continuous, non-invasive, beat-to-beat blood pressure monitoring of a subject, the method comprising:
a. measuring a blood pressure waveform based on an unadjusted hemodynamic signal through use of a sensor (100) coupled to the subject, wherein the unadjusted hemodynamic signal comprises a plurality of heartbeats;
b. deriving an initial systolic blood pressure (SBP) value and an initial waveform contractility value from the blood pressure waveform;
c. deriving, for one or more heartbeats of the plurality of heartbeats, a diastolic transit time (DTT) value, a pulse pressure (PP) value, a raw diastolic blood pressure (DBP) value, an SBP value, and a waveform contractility value from the blood pressure waveform;
d. calculating, for one or more heartbeats of the plurality of heartbeats, one or more estimated DBP values by a predefined formula which is:
$$eDBP ( t ) = {SBP}_{0} - [ {m}_{0} * DTT ( t ) * {( \frac{C ( t )}{{C}_{0}} )}^{- 1} ] , \\ wherein \\ {m}_{0} = \frac{1}{b} * {\sum }_{i = 1}^{b} \frac{{PP}_{0}}{{PP}_{s} ( i )} * \frac{{SBP}_{s} ( i ) - {DBP}_{s} ( i + 1 )}{DTT ( i )} ,$$
wherein
b=heartbeats;
t=time,
SBP=systolic blood pressure,
SBP₀=initial systolic blood pressure,
DTT=diastolic transit time,
C=waveform contractility,
C₀=initial waveform contractility,
PP=pulse pressure,
PP₀=initial pulse pressure, and
DBP=raw diastolic blood pressure;
e. deriving an offset value based on a difference between the one or more estimated DBP values and the one or more raw DBP values;
f. adjusting the blood pressure waveform based on the offset to generate an adjusted blood pressure waveform; and
g. outputting the adjusted blood pressure waveform.

15. The method of claim 14, wherein the calibration factor is calculated over 3 to 10 initial heartbeats of the plurality of heartbeats.

16. The method of claim 14, wherein the sensor (100) comprises a capacitive pressure sensor, a photoplethysmograph sensor, speckleplethysmograph sensor, an optical sensor, a tonometry-based device, or a combination thereof.

17. The method of claim 14, wherein the sensor (100) is communicatively coupled to a computing device (200).

18. The method of claim 17, wherein the sensor (100) is communicatively coupled to the computing device (200) by a wireless component or by a wired component.
